(19) United States
(12) Patent Application Publication
(10) Pub. No.: US 2017/0348253 A1
(43) Pub. Date: Dec. 7, 2017

(54) Treatment of Drug-Resistant Microbial Infections

(71) Applicant: NovaBiotics Limited, Aberdeen (GB)

(72) Inventors: Deborah O'NEIL, Aberdeen (GB); Douglas FRASER-PITT, Aberdeen (GB)

(21) Appl. No.: 15/615,635

(22) Filed: Jun. 6, 2017

Related U.S. Application Data
(60) Provisional application No. 62/346,946, filed on Jun. 7, 2016.

(30) Foreign Application Priority Data
Dec. 16, 2016 (GB) 1621447.0

(51) Int. Cl. A61K 31/145; A61K 31/7052; A61K 31/635; A61K 31/496; A61K 31/546; A61K 31/505; A61K 38/12; A61K 31/7036

(52) U.S. Cl. CPC A61K 31/145; A61K 38/12; A61K 31/7052; A61K 31/7036; A61K 31/496; A61K 31/546; A61K 31/505; A61K 31/635

(57) ABSTRACT

The present invention provides a sulphur-containing compound for use in the prophylaxis or treatment of an infection associated with at least one microbe or microbial strain displaying at least some degree of drug resistance. Also provided is a product comprising a first agent being a sulphur-containing compound and a second agent being an antimicrobial agent, in particular an antibiotic agent.

### CROSS-REFERENCE TO RELATED APPLICATIONS

The present application claims the benefit of U.S. Provisional Application No. 62/346,946, filed on Jun. 7, 2016 and United Kingdom Application No. 1621447.0, filed Dece. 16, 2016, the disclosures of which are incorporated herein by reference in their entirety for all purposes.

### FIELD OF THE INVENTION

The invention relates to the use of a sulphur-containing compound in the treatment of a patient with a drug resistant microbial infection, in particular an antibiotic-resistant infection.

### BACKGROUND TO THE INVENTION

Drug-resistant infectious agents, namely those that are not efficiently killed or substantially growth-inhibited by antimicrobial compounds, are an increasingly important public health concern. Tuberculosis, gonorrhea, malaria and childhood ear infections are examples of diseases which have become more difficult to treat due to the emergence of drug-resistant pathogens. Antimicrobial resistance is becoming a factor in virtually all hospital-acquired (nosocomial) infections. It has been estimated that the annual cost of treating antibiotic resistant infections in the United States alone may be as high as $30 billion.

Resistance has been recognized since the introduction of penicillin nearly 50 years ago, when penicillin-resistant infections caused by Staphylococcus aureus rapidly appeared. Strains of multidrug-resistant tuberculosis (MDR-TB) have emerged over the last decade and pose a particular threat to people infected with HIV. Drug-resistant strains are as contagious as those that are susceptible to drugs. Diarrheal diseases cause almost 3 million deaths a year—mostly in developing countries, where resistant strains of highly pathogenic bacteria such as Shigella dysenteriae, Campylobacter, Vibrio cholerae, Escherichia coli and Salmonella are emerging.

Colistin, the most common polymyxin, is a last-resort treatment for infections with bacteria such as E. coli and Klebsiella that resist all other available antibiotics. Researchers at South China Agricultural University in Guangzhou recently discovered a gene for resistance to colistin in infected livestock, meat and humans (Yi-Yun Liu et al., The Lancet, vol. 16, no. 2, p 161-168, Feb 2016). The mcr-1 gene can pass easily between bacteria. This is particularly concerning in that gram-negative bacteria, which cause common gut, urinary and blood infections in humans, can now become “pan-resistant”, with genes that defeat all antibiotics now available.

Given the escalating problems associated with poorly treatable infections caused by an increasing variety of resistant infectious agents, such as antibiotic-resistant bacteria, there is a great need for improved anti-microbial treatments. The two major avenues for research into such treatments are development of novel antimicrobial compounds and the alternative approach of developing agents which serve to reduce or reverse the resistance displayed by the pathogens. The present inventors have focused on the alternative approach, as disclosed by the present invention.

### STATEMENTS OF THE INVENTION

The present inventors have previously demonstrated the utility of cysteamine as an antimicrobial agent in cystic fibrosis. The present inventors have now shown that cysteamine has broader potential in a wider range of bacterial infections. Surprisingly, the present inventors have now shown that cystearnine has an application in reversing drug resistance and thus improving therapeutic treatment of infections associated with resistant pathogens.

According to a first aspect of the present invention, there is provided a sulphur-containing compound for use in the prophylaxis or treatment of an infection associated with at least one microbe or microbial strain displaying at least some degree of drug resistance. In one embodiment, the infection is a drug-resistant microbial infection. In one embodiment, the infection is a bacterial infection. In a further embodiment in the bacterial infection is an antibiotic resistance bacterial infection.

In a further aspect, the invention provides a prophylactic or curative treatment of an infection associated with at least one microbe or microbial strain displaying at least some degree of antimicrobial resistance comprising administering to a subject a sulphur-containing compound. In one embodiment, the patient is infected with a bacteria which has at least some or some degree of resistance to an antibacterial agent such as an antibiotic. The infection may be an antibiotic-resistant infection. Typically the sulphur-containing compound restores a degree of sensitivity of the antibiotic-resistant infection (or causative bacterium of the infection) to the antibiotic.

According to a further aspect of the present invention there is provided a product comprising a sulphur-containing compound and a second agent being an antimicrobial agent. The sulphur-containing agent and antimicrobial agent, although both have antimicrobial properties, are not the same. According to one embodiment, the antimicrobial agent of the product of the present invention does not comprise peptides. Suitably, the product of the present invention does not comprise peptides.

In a preferred aspect of the invention, the sulphur-containing compound and antimicrobial agent are not administered in a singular dosage unit but are each instead in separate dosage units. The dosage units are preferably administered simultaneously but may be administered in a non-simultaneous fashion due to differential absorption at of the sulphur-containing agent and antimicrobial agent.

In a preferred aspect of the invention, the product is for use in the prophylaxis or treatment of an infection associated with at least one microbe or microbial strain displaying at least some degree of drug resistance.

In a further aspect, the invention provides a prophylactic or curative treatment of an infection associated with at least one microbe or microbial strain displaying at least some degree of antimicrobial resistance comprising administering to a subject a product comprising a sulphur-containing compound and a second agent being an antimicrobial agent. The invention allows for the use of lower amounts of antimicrobials such as antibiotics when combined with a sulphur-containing compound.

A further aspect of the invention provides a kit comprising a first dosage unit comprising a sulphur-containing compound and a further dosage unit comprising an antibacterial agent. In the kit, the antibacterial agent may be an antibiotic.

### DETAILED DESCRIPTION

As used herein “sulphur-containing compound” is intended to cover cysteamine, cystamine or a derivative thereof. The sulphur-containing compound may be an aminothiol. Examples of aminothiols include cysteamine and derivatives thereof. The term “derivative thereof” may encompass 2-methylthio ethylamine (cinnamate), 2-methyl thio ethylurea, N-(2-methylthio ethyl) p-acetamido benzamide, 2-aminoethanethiol, N-(2-methylthio ethyl)p-acetamido benzenesulfonamide,N-(2-propylthioethyl)-p-methoxy benzamide, N-(butylthio ethyl) nicotinamide, N-(2-dodecylthio ethyl) p-butoxybenzamide, N-(2-methylthio ethyl) p-toluenesulfonamide, N-(2-isopropylthio ethyl) propionamide, N-(2-octylthio ethyl) acetamide, N-(2-butylthio ethyl) methanesulfonamide, N-(2-isopentylthioethyl)butane, bis 1,4-(2-acetamido ethylthio), 2,3-butanediol, 2-hexadecylthio ethylamine hydrochloride, 2-allylthio ethylamine malate,9-octadecene 2-ylthio ethylamine hydrochloride, 2-dodecylthio ethylamine hydrochloride, 2-isopentylthio ethylamine mandelate, 2-octadecylthio ethylamine salicylate, 2-beta-hydroxyethyl thio ethylurea, 2-beta-hydroxyethylthio ethylamine hydrochloride, 2-(2,3 -dihydroxy propylthio)ethylamine p-toluenesulfonate, 2-(2-hydroxypropylthio)ethylamineoxalate, N-(2-methylthio ethyl)phenylacetamide, 2-(2,2-dimethoxy ethylthio) ethylamine hydrochloride, 2-(2,2-dimethoxy ethylthio) ethylamineundecylenate, 2-(2,2-diethoxy ethylthio) ethylamine undecylenate, 2-(2,2-diethoxy ethylthio)ethylamine acetate, 2-undecenylthio ethylamine, 2-beta-ureidoethylthio ethylamine hydrochloride, 2-beta-acetamidoethylthio ethylamine tropate, 2,2′-thio diethylamine fumarate, 2,2′-thio diethylurea, 3-beta-aminoethylthio propylamine hydrochloride, S-beta-ureidoethyl thiocarbamate, 2-ethoxycarbonylthio ethylamine hydrochloride, 2-dimethylamino carbonylthio ethylamine sulfate, 2-butoxycarbonyl methylthio ethylurea, 2-ethyloxycarbonylmethylthio ethylamine hydrochloride, 6-beta-aminoethylthio hexanoate of methyl hydrochloride, 5-beta-aminoethylthio pentanoic acid, 2-phenylthio ethylamine dihydrogen phosphate, 2-p-t-butylphenylthio ethylamine trichloracetate, 2-p-methoxyphenylthio ethylamine ditartrate, 2-tolylthio ethylamine hydrobromide, 2-(1-biphenyl thio) ethylamine hydrochloride, 2-N-pentachlorophenylthio ethyl acetamide, 2-benzylthio ethylamine malate, 2-benzylthio ethylamine nicotinate, 2-benzylthio 2-methyl propylamine hydrochloride, 2-benzylthio propylamine lactate, N-(2-benzylthio ethyl)nicotinamide hydrochloride, N-(2-benzylthio ethyl) 10-undecene amide, N-(2-benzylthio ethyl) hexadecanamide, S-beta-aminoethyl mercaptobutyric acid, N-(2-benzylthio ethyl)formamide, N-(2-benzylthio ethyl)phenylacetamide, N-[2-(2,6-dimethyl pheny)ethyl] hexanamide, 2-o-aminophenylthio ethylamine succinate, N-(2-benzylthio ethyl) glutamine, S-beta-aminoethyl mercapto acetic acid (3-S-beta-aminoethyl) mercapto propionic acid, (3-S-.gamma.-amino propyl) mercapto acetic acid, S(2-p-methoxybenzamido ethyl) mercapto 2-(2-naphtyl methylthio) ethylamine hydrochloride, 2-(2-naphtyl methylthio) ethylamine disuccinate, (2-thenyl) 2-thio ethylamine hydrobromide, 2-N-acetyl (2-thenylthio-ethylamine, 2-o-chlorobenzylthio ethylamine hydrochloride, 2-p-chlorobenzylthio ethylamine glycolate, 2-o-fluorobenzylthio ethylamine hydrochloride, 2-furfurylthio ethylamine hydrochloride, 2-tetrahydrofurfurylthio ethylamine p-amino-benzoate, 2-beta-phenylethylthio ethylamine glutamate, 2-diphenylmethylthio ethylamine hydrochloride, 2-triphenyl methylthio ethylamine hydrochloride hemihydrate, 2-(2-pyridyl ethylthio)ethylamine hydrochloride, 2-(2-p-toluene sulfonamido ethylthio) pyridine N-oxide, 2-beta-aminoethylthiomethyl pyridine N-oxide dihydrochloride, 2-beta-aminoethylthio pyridine N-oxide hydrochloride, 2,4-dichloro 2-benzylthio ethylamine aspartate, N-[2-(3,4-dichloro benzylthio)ethyl] butyramide, N-[2-(2,6-dichloro benzylthio)ethyl] dodecanamide, N-[2-(3,5-dichloro benzylthio)ethyl] trifluoroacetamide hydrochloride, 2-p-ethoxybenzylthio ethylamine hydrochloride, N-[2-m-fluorobenzylthio ethyl] chloroacetamide, 2-p-bromobenzylthio ethylamine succinate, 2-(3,4-dimethoxy benzylthio)ethylamine malate, 2-(3,4-methylenedioxy benzylthio)ethylamine hydrochloride, 2-(2,4-dichloro cetylthio)ethylamine, 2 (3,4,5-trimethoxy benzylthio)ethylamine hydrocinnamate, 2-p-methoxy benzylthio ethylamine salicylate, 2-o-methylbenzylthio ethylamine phenyl-acetate, N-[2-p-dimethylaminobenzylthio ethyl] methane-sulfonamide,2-p-phenoxybenzylthio ethylamine hydrochloride, 2-beta-aminoethylthio pyridine hydrochloride, 2-benzylthio ethylamine citrate, N-[2-benzylthio ethyl] 2,4-dihydroxy 3,3-dimethyl butyramide, N-(2-benzylthio ethyl) 6,8-dihydroxy 7,7-dimethyl 5-oxo 4-aza octanamide, N-[2-(2-pyridyl thio)ethyl] propionamide, 2-(2-pyridyl methylthio)ethylamine dihydrochloride, 2-benzylthio ethylamine pantothenate, S-(beta-acetamidoethyl)mercapto acetate of beta.-morpholinoethyl, S-(beta-phenylacetamidoethyl)mercaptoacetate N′-methyl 2-piperazino ethyl, S-(beta-ureidoethyl)mercaptoacetate of beta.-pyrrolidino-ethy, S -(beta-trifluoroacetamidoethyl)-betamercapto-propionate of beta-dimethylaminoethyl, 2-p-nitrobenzylthio ethylamine crotonate, 2-beta-morpholinocarbonyl ethylthio ethylamine hydrochloride, N,N-di(hydroxyethyl)S-(beta-benzamido-ethyl) mercapto-acetamido, N[2-N′-methyl piperazino carbonylthio ethyl] acetamide, 2-(1-naphthyl thio)ethylamine hydrochloride, N-(3 -beta-ureidoethylthio propyl) succinamic acid, 3-allylthio propylamine, 3-(2,2′-dimethoxy ethylthio)propylamine, 3-(2,2′-dimethoxy ethylthio)propylamine sulfate, S-beta-aminoethylmercapto acetic acid, the hydrochloride of S-beta-aminoethyl mercapto acetic acid, N-(2-benzylthioethyl)acetamide, N-(2-benzylthioethyl)propionamide, N-(2-benzylthioethyl)butyramide, N-(2-benzylthioethyl)methanesulfonamide, N-(2-benzylthioethyl)ethanesulfonamide, N-(2-benzylthioethyl-propanesulfonamide, N-(2-benzylthioethyl)butanesulfonamide, S-(2-p-acetamidobenzenesulfonamido ethyl) mercapto acetic acid, S-(2-p-acetamidobenzamido ethyl) mercapto acetic acid, N-(2-thenylthioethyl)acetamide, 2-benzylthio propylamine, 2-benzylthio 2-methyl propylamine, 2-(2-p-toluenesulfonamido ethylthio) pyridine N-oxide, S-(2-p-butoxybenzamidoethyl)mercapto acetic acid, 2-t-butylthio ethylamine hydrochloride, 2-methoxy carbonyl methylthio ethylamine hydrochloride, 2-ethoxycarbonylmethylthio ethylamine hydrochloride, 2-propoxycarbonylmethyl thio ethylamine hydrochloride, 2-butoxycarbonylmethylthio ethylamine hydrochloride, 2,2′-thio diethylamine dihydrochloride, 3 -(2-aminoethylthio)alanine hydrochloride, 2-benzylthio ethylammonium diacid phosphate, 2-methylthio ethylamine, N-(methylthioethyl) p-acetamidobenzamide, N-(2-methylthioethyl)nicotinamide, N-(2-methylthioethyl)benzamide, N-(2-methylthioethyl) p-butoxybenzamide, N-(2-methylthioethyl) butyramide, N-(2-methylthioethyl) propionamide, N-(2-methylthioethyl) acetamide, N-(2-methylthioethyl) butanesulfonamide, N-(2-octylthioethyl) methanesulfonamide, 2-cetylthio ethylamine hydrochloride, 2-(2-hydroxyethylthio) ethylamine hydrochloride, 2-methylthio ethylamine phenylacetatesnd 2-methylthio ethylamine undecylenate.

Alternatively, the sulphur-containing compound may be an organic disulphide, such as cystamine.

The sulphur-containing compound of the invention may be administered in the form of pharmaceutically acceptable salts. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., US, 1985, p. 1418, the disclosure of which is hereby incorporated by reference; see also Stahl et al, Eds, “Handbook of Pharmaceutical Salts Properties Selection and Use”, Verlag Helvetica Chimica Acta and Wiley-VCH, 2002. The phrase “pharmaceutically acceptable” is employed herein to refer to those compounds, materials, compositions, and/or dosage forms which are, within the scope of sound medical judgment, suitable for use in contact with the tissues of human beings or, as the case may be, an animal without excessive toxicity, irritation, allergic response, or other problem or complication, commensurate with a reasonable benefit/risk ratio.

The invention thus includes pharmaceutically-acceptable salts of the disclosed compounds wherein the parent compound is modified by making acid or base salts thereof for example the conventional non-toxic salts or the quaternary ammonium salts which are formed, e.g., from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

The terms “antimicrobial resistance” and “resistance” are used interchangeably to describe a situation where a pathogenic microbe has undergone some sort of change that reduces or eliminates the effectiveness of drugs, chemicals, or other agents to cure or prevent infections.

The terms “microbes” is used in its common meaning, i.e. to cover pathogenic organisms so small that a microscope is required to see them. Microbes are also called microorganisms, and include bacteria, viruses, fungi, and parasites, out of which the former two, especially bacteria are the most relevant for the purposes of the present in vention.

As used herein, the term “antimicrobial agent” is intended to cover drugs, chemicals, or other substances that either kill or slow the growth of microbes. Among the antimicrobial agents in use today are antibacterial drugs, antiviral agents, antifungal agents, and antiparasitic drugs.

Preferably the antimicrobial agent is an antibacterial agent, for example an antibiotic agent. Antibiotic agents may be bactericidal and/or bacteriostatic.

The antibiotic agent may contain a β-lactam ring. The β-lactam ring is part of the core structure of several antibiotic families, the principal ones being the penicillins, cephalosporins, carbapenems, and monobactams. These antibiotic agent are called β-lactam antibiotics.

Generally the antibiotic agent is of the group consisting of aminoglycosides, ansamycins, carbacephem, β-lactams carbapenems, cephalosporins, (including first, second, third, fourth and fifth generation cephalosporins), penicillin, monobactams), glycylcyclines, lincosamides, lipopeptides, macrolides, nitrofurans, oxazolidinones, quinolones, sulfonamides, polypeptides and tetracyclins.

The antibiotic agent may be of the group consisting of aminoglycosides, ansamycins, carbacephem, carbapenems, cephalosporins (including first, second, third, fourth and fifth generation cephalosporins), lincosamides, macrolides, monobactams, nitrofurans, quinolones, penicillin, sulfonamides, polypeptides and tetracyclins. Alternatively or additionally the antibiotic agent may be effective against mycobacteria.

The antibiotic agent may be an aminoglycoside such as Amikacin, Gentamicin, Kanamycin, Neomycin, Netilmicin, Tobramycin or Paromomycin.

The antibiotic agent may be an Ansamycin such as Geldanamycin and Herbimycin

Alternatively the antibiotic agent may be a carbacephem such as Loracarbef.

The antibiotic agent is a carbapenem such as Ertapenem, Doripenem, Imipenem/Cilastatin or Meropenem.

Alternatively the antibiotic agent may be a cephalosporins (first generation) such as Cefadroxil, Cefazolin, Cefalexin, Cefalotin or Cefalothin, or alternatively a Cephalosporins (second generation) such as Cefaclor, Cefamandole, Cefoxitin, Cefprozil or Cefuroxime. Alternatively the antibiotic agent may be a Cephalosporins (third generation) such as Cefixime, Cefdinir, Cefditoren, Cefoperazone, Cefotaxime, Cefpodoxime, Ceftibuten, Ceftizoxime and Ceftriaxone or a Cephalosporins (fourth generation) such as Cefepime and Ceftobiprole.

The antibiotic agent may be a lincosamides such as Clindamycin and Azithromycin, or a macrolide such as Azithromycin, Clarithromycin, Dirithromycin, Erythromycin, Roxithromycin, Troleandomycin, Telithromycin and Spectinomycin.

Alternatively the antibiotic agent may be a monobactams such as Aztreonam, or a nitrofuran such as Furazolidone or Nitrofurantoin.

The antibiotic agent may be a penicillin such as Amoxicillin, Ampicillin, Azlocillin, Carbenicillin, Cloxacillin, Dicloxacillin, Flucloxacillin, Mezlocillin, Nafcillin, Oxacillin, Penicillin G or V, Piperacillin, Temocillin and Ticarcillin.

The antibiotic agent may be an oxazolidinone such as linezolid or tedizolid.

The antibiotic agent may be a sulfonamide such as Mafenide, Sulfonamidochrysoidine, Sulfacetamide, Sulfadiazine, Silver sulfadiazine, Sulfamethizole, Sulfamethoxazole, Sulfanilimide, Sulfasalazine, Sulfisoxazole, Trimethoprim, and Trimethoprim-Sulfamethoxazole (Co-trimoxazole) (TMP-SMX).

The antibiotic agent may be a quinolone such as Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin and Temafloxacin.

The antibiotic agent may be a polypeptide. Examples of such polypeptides include Bacitracin, Colistin and Polymyxin B. In one embodiment, the antibiotic agent is not a polypeptide.

The antibiotic agent may be a lipopeptide. Examples of such lipopeptides include Daptomycin and Surfactin.

Alternatively, the antibiotic agent may be a tetracycline such as Demeclocycline, Doxycycline, Minocycline and Oxytetracycline.

Alternatively the antibiotic agent may be a glycylcycline. Examples of such glycylcyclines include tigecycline.

Alternatively or additionally the antibiotic agent may be effective against mycobacteria. In particular the antibiotic agent may be Clofazimine, Lamprene, Dapsone, Capreomycin, Cycloserine, Ethambutol, Ethionamide, Isoniazid, Pyrazinamide, Rifampicin, Rifabutin, Rifapentine or Streptomycin.

In one embodiment, the antibiotic agent is a macrolide and/or an aminoglycoside and/or sulphonamides.

In one embodiment, the antibiotic agent is a macrolide and/or an aminoglycoside.

In one embodiment, the antibiotic agent is a macrolide and/or sulphonamide.

In one embodiment, the antibiotic agent is an aminoglycoside and/or sulphonamide.

In one embodiment, the antibiotic is selected from tobramycin, azithromycin, telithromycin, ciproflaxin, ceftazidime.

In one embodiment, the antibiotic agent is not ciproflaxin. In another embodiment the antibiotic is not tobramycin.

The antibiotic agent may be active in the treatment or prophylaxis of infections caused by Enterobacteriaceae (e.g. E.coli or Klebsiella spp., such as K. pneumoniae) or non-Enterobacteriaceae bacteria such as Burkholderia spp.

Generally the antibiotic agent is active in the treatment or prophylaxis of infections caused by gram-negative or gram-positive bacteria, such as Pseudomonas spp., for example Pseudomonas aeruginosa, Burkholderia spp., Escherichia coli, Klebsiella spp., for example K. pneumoniae, Staphylococcus spp., for example S. aureus.

In one embodiment of the invention, the antibiotic is not a β-lactam antibiotic.

In one embodiment of the invention, the antibiotic is not a penicillin.

In one embodiment of the invention, the antibiotic is not a cephalosporin.

In one embodiment of the invention, the antibiotic is not a carbapenem.

In one embodiment of the invention, the antibiotic is not a monobactam.

In one aspect of the invention the normal dosing regime of the antibiotic can be reduced by up to 10%; such as by up to 20%; such as by up to 30%; such as by up to 40%; such as by up to 50%; such as by up to 55%. Thus the dosing of an antibiotic in the present invention can be reduced, according to the dose provided by the MIC values exemplified in present invention.

In another aspect of the invention the risk of developing antibiotic resistance can be dramatically reduced. By combining the active principle of antibiotics with a sulphur-containing compound such as cysteamine, the effect of the antibiotic may be increased up to 10 times giving two alternative advantages; the micro-organisms are up to 10 times as susceptible increasing the efficiency of the therapy; alternatively the therapeutic dose can be reduced. concurrently with 90% while maintaining the therapeutic effect.

In a preferred aspect of the invention, the sulphur-containing compound and the additional antimicrobial agent may be administered simultaneously, sequentially or separately. The sulphur-containing compound and the additional antimicrobial agent may be provided as a combination package. The combination package may further instructions for simultaneous, separate or sequential administration of each of the sulphur-containing compound and additional antimicrobial agent. For sequential administration, the sulphur-containing compound and the additional antimicrobial agent may be administered in any order. In one embodiment, the sulphur-containing compound is administered before the additional antimicrobial agent.

### PHARMACEUTICAL PRODUCT

The present invention provides a product comprising a first active agent being sulphur-containing compound and a second agent being an antimicrobial agent.

The above mentioned active agents may be administered as free or fixed combinations. Free combinations may be provided as combination packages containing all the active agents in free combinations. Fixed combinations are often tablets or capsules.

The active agents may be administered simultaneously, sequentially or separately. The active agents may be provided as a combination package. The combination package may contain the product of the invention together with instructions for simultaneous, separate or sequential administration of each of the active agents. For sequential administration, the active agents can be administered in any order.

The active agents of the product of the invention may be provided as pharmaceutical compositions additionally containing one or more pharmaceutically acceptable diluents, excipients and/or carriers. This applies to both fixed and free combinations.

The active agents of the present invention may be administered by any suitable route known to those skilled in the art, preferably in the form of a pharmaceutical composition adapted to such a route, and in a dose effective for the treatment intended. The active compounds and composition may, for example, be administered parenterally, orally, intranasal, intrabronchial, enterally, transdermally, sublingually, rectally, vaginally, ocularly, or topically. Both local and systemic administration is contemplated.

For the purposes of parenteral administration (“parenteral” as used herein, refers to modes of administration which include intravenous, intramuscular, enteral, intraperitoneal, intrasternal, subcutaneous and intraarticular injection and infusion of which intravenous (including continuous intravenous administration) is most preferred) solutions in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

The sulphur-containing compound may be administered parenterally before parenteral administration of the additional antimicrobial agent. Alternatively, the sulphur-containing compound may be administered parenterally simultaneously with parenteral before administration of the additional antimicrobial agent.

The products of the invention can also be administered intranasally or by inhalation and are conveniently delivered in the form of a dry powder inhaler or an aerosol spray presentation from a pressurised container, pump, spray, atomiser, nebuliser, with or without the use of a suitable propellant.

Alternatively the products of the invention can be administered in the form of a suppository or pessary, or they may be applied topically in the form of a gel, hydrogel, lotion, solution, cream, ointment or powder. The products of the invention may be dermally or transdermally administered, for example, by use of a skin patch, depot or subcutaneous injection. They may also be administered by pulmonary or rectal routes.

For oral administration, the pharmaceutical composition may be in the form of; for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose; mannitol, corn starch or potato starch; with binders such as crystalline cellulose, cellulose derivatives, acacia, corn starch or gelatins; with disintegrators such as corn starch, potato starch or sodium carboxymethylcellulose; and with lubricants such as talc or magnesium stearate. The active ingredient may also be administered by injection as a composition wherein, for example, saline, dextrose or water may be used as a suitable carrier.

The products of the invention may also find application as/in an oral formulation wherein the product is formulated in a carrier, for example selected from films, tapes, gels, microspheres, lozenges, chewing gum, dentrifices and mouthwash.

The amount of therapeutically active compound that is administered and the dosage regimen for treating a disease condition with the compounds and/or compositions of this invention depends on a variety of factors, including the age, weight, sex and medical condition of the subject, the severity of the disease, the route and frequency of administration, and the particular compound employed, as well as the pharmacokinetic properties of the individual treated, and thus may vary widely. The dosage will generally be lower if the compounds are administered locally rather than systemically, and for prevention rather than for treatment. Such treatments may be administered as often as necessary and for the period of time judged necessary by the treating physician. One of skill in the art will appreciate that the dosage regime or therapeutically effective amount of the inhibitor to be administrated may need to be optimized for each individual. The pharmaceutical compositions may contain active ingredient in the range of about 0.1 to 2000 mg, preferably in the range of about 0.5 to 500 mg and most preferably between about 1 and 200 mg. A daily dose of about 0.01 to 100 mg/kg body weight, preferably between about 0.1 and about 50 mg/kg body weight and most preferably from about 1 to 20 mg/kg body weight, may be appropriate. The daily dose can be administered in one to four doses per day.

The products of the invention may be administered to the respiratory tract. Thus, the present invention also provides aerosol pharmaceutical formulations comprising a product of the invention. Also provided is a nebuliser or inhaler containing a product of the invention.

Additionally, the products of the invention may be suited to formulation as sustained release dosage forms and the like. The formulations can be so constituted that they release the active agents, for example, in a particular part of the intestinal or respiratory tract, possibly over a period of time. Coatings, envelopes, and protective matrices may be made, for example, from polymeric substances, such as polylactide-glycolates, liposomes, microemulsions, microparticles, nanoparticles, or waxes. These coatings, envelopes, and protective matrices are useful to coat indwelling devices, e.g. stents, catheters, peritoneal dialysis tubing, draining devices and the like.

### METHODS AND USE

The invention provides the use of a sulphur-containing compound in the prophylaxis or treatment of an infection associated with at least one microbe or microbial strain displaying at least some degree of drug resistance. In one embodiment, the infection is a drug-resistant microbial infection. In one embodiment, the infection is a bacterial infection. In a further embodiment in the bacterial infection is an antibiotic resistance bacterial infection.

The invention also provides a prophylactic or curative: treatment of an infection associated with at least one microbe or microbial strain displaying at least some degree of antimicrobial resistance comprising administering to a subject a sulphur-containing compound.

The bacterial infection may include an infection caused by more than one microorganism, for example bacteria and any one of fungi, yeast, viruses and protozoa.

The bacterium may be a Gram-positive or a Gram-negative bacterium. A bacterial pathogen may be derived from a bacterial species selected from the group consisting of:Staphylococcus spp., e.g. Staphylococcus aureus such as Methicillin resistant S. aureus, Staphylococcus epidermidis; Enterococcus spp., e.g. Enterococcus faecalis; Streptococcus pyogenes; Listeria spp.; Pseudomonas spp.;Mycobacterium spp., e.g. Mycobacterium tuberculosis; Enterobacter spp.; Campylobacter spp.; Salmonella spp.; Streptococcus spp., e.g. Streptococcus Group A or B, Streptoccocus pneumoniae; Helicobacter spp., e.g. Helicobacter pylori; Neisseria spp., e.g. Neisseria gonorrhea, Neisseria meningitidis; Borrelia burgdorferi; Shigella spp., e.g. Shigella flexneri; Escherichia coli; Haemophilus spp., e.g. Haemophilus influenzae; Chlamydia spp., e.g. Chlamydia trachomatis, Chlamydia pneumoniae, Chlamydia psittaci; Francisella fularensis; Bacillus spp., e.g. Bacillus anthraces; Clostridia spp., e.g. Clostridium botulinum; Yersinia spp., e.g. Yersinia pestis; Treponema spp.; Burkholderia spp.; e.g. Burkholderia mallet and B pseudomallei.

In particular the bacterium may include Pseudomonas spp., for example Pseudomonas aeruginosa; Staphylococcus spp., for example Staphylococcus aureus and Staphylococcus epidermidis; Haemophilus spp., for example Haemophilus influenza; Burkholderia spp., for example Burkholderia cepacia; Streptococcus spp., Propionibacterium spp., for example Propionibacterium acnes. Preferably the bacterium is selected from Pseudomonas spp., for example Pseudomonas aeruginosa and Staphylococcus spp., for example Staphylococcus aureus and Staphylococcus epidermidis.

In one embodiment of the invention, the bacterial infection is caused by Enterobacteriaceae (e.g. E.coli or Klebsiella spp., such as K. pneumoniae) or non-Enterobacteriaceae bacteria such as Burkholderia spp., for example B.cepacia or B.multivorans.

In a further embodiment of the invention, the bacterial infection is caused by gram-negative or gram-positive bacteria, such as Pseudomonas spp., for example Pseudomonas aeruginosa, Burkholderia spp., Escherichia coli, Klebsiella spp., for example K. pneumoniae, staphylococcus spp., for example S. aureus, in particular Methicillin resistant S.aureus.

In one embodiment of the invention, the bacterial infection is caused by a bacterium no including Burkholderia spp. In another embodiment of the invention, the bacterial infection is caused by a bacterium not including Pseudomonas spp. for example Pseudomonas aeruginosa.

The method of the invention may be used to minimise and prevent the formation of bacterial colonies, in particular bacterial biofilms in a variety of environments including, but not limited to, household, workplace, laboratory, industrial environment, aquatic environment (e.g., pipeline systems), medical devices including indwelling devices such as defined herein, dental devices or dental implants, animal body for example human body.

The method of the invention may be used to prevent or restrict the formation of a bacterial colony. The method of the present invention may be used to prevent or treat bacterial infections including topical infections, oral infections and systemic infections. Topical infections may include wounds, ulcers and lesions for example, cutaneous wounds such cuts or burns, and conditions associated therewith.

Oral infections may include gingivitis, periodontitis and mucositis.

Systemic infections may include cystic fibrosis, COPD and other conditions associated with mucosal infections, for example, gastrointestinal, urogenital or other respiratory infections.

The product of the invention may be useful in the prevention of, delay of progression of, or treatment of a disease or condition selected from the group consisting of skin and wound infections, middle-ear infections, gastrointestinal tract infections, peritoneal membrane infections, urogenital tract infections, oral soft tissue infections, formation of dental plaque, eye infections (including contact lens contamination), endocarditis, infections in cystic fibrosis, and infections of indwelling medical devices such as described herein.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### BRIEF DESCRIPTION OF THE DRAWINGS

The invention will now be described by way of Examples only with reference to the following Figures in which:

FIG. 1 is a graph showing that cysteamine chemopotentiates the activity of ciprofloxacin in the neutropenic mouse thigh model of infection with P. aeruginosa LES431. Legend::1. Vehicle control, 2. Colistin [5 mg/kg] (positive control), 3. Cysteamine only [1.25 mg/kg], 4. Ciprofloxacin only [15 mg/kg], and 5. Ciprofloxacin+Cysteamine. One way Anova with Tukey's post hoc test analysis. ***=p<0.001, **=p<0.01, ns=not significant.

### EXAMPLES

### Methods

### MIC₁₀₀ Determination

The MIC₁₀₀ (concentration at which 100% of bacteria were killed) of all Burkholderia cepacia complex (Bcc) isolates was determined versus cysteamine and the antibiotics tobramycin, ciprofloxacin, ceftazidime and trimethoprim/sulfamethoxazole using the CLSI broth microdilution procedure (LSI. 2012a. Methods for dilution antimicrobial susceptibility tests for bacteria that grow aerobically; Approved Standard-Ninth Edition; M07-A9. Wayne, Pa.: Clinical and Laboratory Standards Institute).

MIC₁₀₀ values for Burkholderia strains are described as the concentration of antibiotic (in micrograms/ml) required to inhibit the growth of all of the bacteria tested, cultured over 48 hours at 37° C. in cation-adjusted Müller-Hinton broth. The initial inoculum is prepared from a single colony recovered from frozen stocks (after confirmation of culture purity) which is transferred via sterile inoculation loop to 10 ml cation-adjusted Müller-Hinton broth in a 30 ml universal container. This is incubated aerobically and statically for 48 h at 37° C. prior to the experiment.

The inoculum is standardised by comparison with 0.5 McFarland standard absorption at 625 nm. This is done by serially diluting 100 μl of the liquid culture 2-fold in phosphate buffered saline on a 96-well microtitre plate and comparing with the mean value of triplicate 100 μl volumes of 0.5 McFarland standards on the same plate. The closest dilution is then diluted a further 1:150 in sterile, twice-concentrated, cation-adjusted Müller-Hinton broth.

In the test plate, antibiotics of choice (tobramycin, ciprofloxacin, ceftazidime and trimethoprim/sulfamethoxazole) are serially diluted 2-fold in sterile distilled water to achieve 2x the relevant concentrations used in each experiment, by diluting 50 μl of each antibiotic into 50 μl volumes of distilled water. Negative controls contain 50 μl of sterile distilled water only.

To these plates 50 μl of 1:150 diluted inoculum is added to appropriate wells. Negative culture controls are also prepared by the addition of 50 μl of sterile cation-adjusted Müller-Hinton broth. This brings the final concentration of Müller-Hinton broth to 1×in 100 μl volumes in each well each containing the required concentration of test antibiotic. Typically, each 96-well plate will contain 3 experimental replicates for each Burkholderia strain at each concentration of antibiotic.

The plates are then read at 625 nm using the Biotek plate reader to obtain a time 0 h baseline absorbance reading. The plates are then incubated for 48 h at 37° C. At 48 h the plates are read at 625 nm using the Biotek plate reader to determine growth of bacteria over time in relevant concentrations of test antibiotic. Mean absorbance values are calculated using Microsoft Excel and base line optical density values taken at time 0 h are subtracted. The concentration of antibiotic required for complete inhibition of bacterial growth (MIC₁₀₀) is determined as the concentration with absorbance the same as or below those for the uninoculated controls.

### Checkerboard Assays

Checkerboard assays of cysteamine and antibiotics were conducted according to the method of Burkhart, et al 2006. Antibiotic susceptibility profiling of Bcc (resistant, intermediate or sensitive to antibiotics) was performed using CLSI Performance Standards for Antimicrobial Susceptibility Testing using other non-Enterobacteriaceae interpretive standards, Wayne Pa., 2012b.

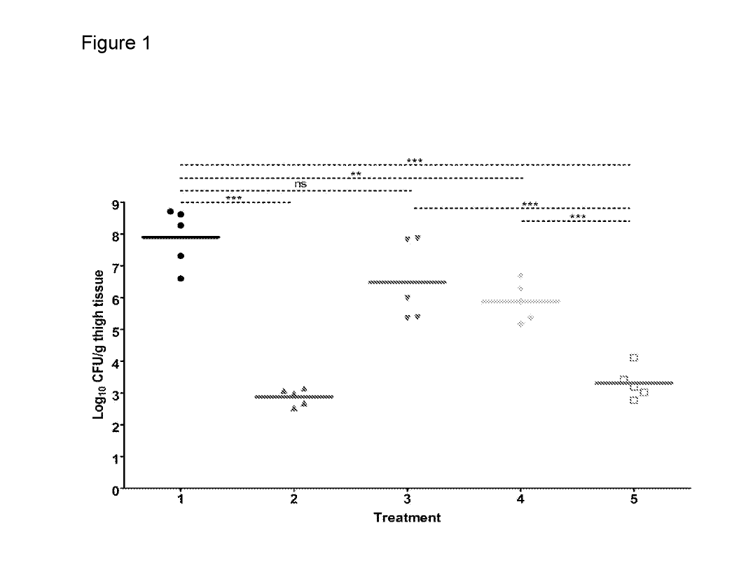

This involves combining two antibiotic dilution series on one 96-well microtitre plate to assess the effect of co-therapy on the growth of microorganisms determined by optical density at 625 nm, and was adapted from the Burkhart et al., method mentioned above. A typical plate plan is illustrated in FIG. 1 below. Antibiotics are prepared in two separate, sterile, 96-well microtitre plates at 4× the final concentration by two-fold serial dilutions in sterile distilled water across the plates in different directions. Water only is added to the negative inoculum and no antibiotic controls. Various volumes may be used to perform the serial dilutions depending upon the amount of antibiotic required for the challenge experiments if performing multiple experimental replicates, or preparing the same antibiotic to challenge a number of different strains of Bcc. A total of 25 μl of each dilution of antibiotic and controls is required for each challenge plate, so (for example) a two-fold serial dilution of antibiotic made using 150 μl volumes would be (in theory) enough volume for six challenge plates (150±25=6), although in practice, due to volume retention by pipette tips this should be adequate for 5 challenge plates. Antibiotic plates can be prepared in advance of the experiment, depending upon the stability of the antibiotic used, by performing the dilutions and freezing the plates at -20° C. prior to the day of the experiment.

On the day of the experiment, 25 μl from each well on the antibiotic plates is transferred to the challenge plate giving a total volume of 50 μl. Negative, uninoculated, controls are prepared by adding 50 μl of cation-adjusted Muller Hinton broth. Incolum are prepared as described above, using the referenced method (LSI. 2012a. Methods for dilution antimicrobial susceptibility tests for bacteria that grow aerobically; Approved Standard-Ninth Edition; M07-A9. Wayne, Pa.: Clinical and Laboratory Standards Institute). Cultures (grown previously for 48 h in cation-adjusted Müller Hinton broth at 37° C.) are standardised by comparing to a 0.5 McFarland standard optical density at 625 nm using a Biotek plate reader. The appropriate dilution is then further diluted 1:150 in twice-concentrated cation-adjusted Müller Hinton broth. Fifty microliters of this dilution is used to inoculate the plate to give final volumes of 100 μl per well of 1×cation-adjusted Müller Hinton broth and the appropriate concentrations of each antibiotic.

A time 0 h reading is then taken at 625 nm using the Biotek plate reader to determine background absorbance. The plates are then incubated, statically, for a further 48 h at 37° C. prior to another reading at 625 nm to determine growth and the efficacy of antibiotic co-therapy.

From each plate it is possible to determine the MIC₁₀₀ for each antibiotic when used alone (single antibiotic controls), as well as the MIC₁₀₀ for antibiotics in combination. This method is also used to calculate the Fractional Inhibitory Concentration Index (FICI) using the formula shown below but for our purposes we wanted to determine if co-therapy improved the efficacy of the clinically defined antibiotic. Using interpretive criteria defined by CLSI (CLSI Performance Standards for Antimicrobial Susceptibility Testing using Other non-Enterobacteriaceae interpretive standards, Wayne Pa., 2012b), we could determine if a strain was defined as resistant, intermediate or sensitive to monotherapy with the antibiotics tested using MIC₁₀₀ testing methods described above, as well as from single antibiotic control dilutions in the checkerboard experiments. The checkerboard experiments could then be used to determine if co-therapy altered sensitivity or reversed resistance to the clinical antibiotic as defined by CLSI interpretive criteria.

Cloning of mcr-1 gene for phosphoethanolamine-mediated colistin resistance into E. coli NEB® Express laboratory protein expression strain

The open reading frame sequence for the gene mcr-1, a probable phosphoethanolamine transferase (accession number A0A0R6L508) was synthesised using the GeneArt gene synthesis service (Thermo Fisher Scientific). This sequence was amplified using the polymerase chain reaction (PCR) with flanking primers and digested with appropriate restriction enzymes (Ndel and Xhol) and ligated, in-frame, into the multiple cloning site of plasmid pET29b. Plasmids with, and without, mcr-1 insert were transformed into E. coli NEB® Express laboratory strain of E. coli. Internal detection primers were used to confirm presence or absence of the mcr-1 insert in the transformed cells and expression of the mcr-1 gene was confirmed due to phenotypic change in the MIC of this strain to colistin (using the method as described above).

### Etest® Assessment of Antibiotic MIC of Neisseria Gonorrhoeae

GC agar plates were prepared containing Vitox supplement (Oxoid™ Thermo Fisher Scientific, Mass., USA) with and without a range of concentrations of filter-sterilised cysteamine. This is done by autoclaving the required volume of GC agar in solution at 121° C. for 15 min. Following this the agar is allowed to cool to 60° C. prior to the addition of vitox supplement (for the cultivation of fastidious N. gonorrhoeae) and cysteamine solutions as required to final concentration ranges of 0, 128, 256, and 512 mg/L in 100 ml volumes. The agar is then poured, evenly, under aseptic conditions and the plates are allowed to set and dry in a safety cabinet or lamina flow hood.

N. gonorrhoeae is prepared by culturing overnight at 37° C. in a 5% CO₂ atmosphere on GC agar plus vitox plate. A suspension of N. gonorrhoeae cells is made by aseptically transferring loops of overnight growth from the surface of the GC agar plus vitox plate into sterile PBS under aseptic conditions. This is then serially diluted two-fold in PBS to reach the required optical density by comparison to 0.5 McFarland standard. A sterile swab is then inoculated with the appropriate dilution of suspended cells in PBS and this is used to create a spread plate by streaking over the full surface of the appropriately labelled plate and allowed to absorb.

As appropriate, an Etest® strip, containing a standardised gradient of antibiotic is placed (using sterile forceps) onto the surface of inoculated plate. Plates are then incubated upside down at 37° C. in a 5% CO₂ atmosphere for 24 hrs to allow growth of N. gonorrhoeae and the appearance of a zone of clearance on each plate (where this occurs). The MIC can be determined by the point at which along this gradient strip no further growth occurs leading to a zone of clearance on the surface of the agar. Experiments were conducted in triplicate on 3 separate occasions.

### Mouse Thigh Model of Infection

Mouse thigh infection model experiments were conducted at Eurofins Panlabs, (Taipei, Taiwan). Mice were rendered neutropoenic with cyclophosphamide prior to infection. They were separated into groups of 5 animals per treatment. Inoculation (confirmed by culture) was with 1.52×10⁶ cfu/ml of P. aeruginosa strain LES431 and conducted 1 hour prior to treatment which was i.v. for saline vehicle controls, ciprofloxacin, and cysteamine treated mice, and SC for colistin (used as a positive control). Animals were sacrificed 25 h post infection (24 h post-treatment) and thigh weights and cfu/g of tissue were calculated and recorded.

### Microbiology From Aberdeen Clinical Study

Patients≧18 years of age, weighing>50 kg with stable CF lung disease were commenced on oral cysteamine bitartrate (Cystagon®) 450 mg once daily, increased weekly to 450 mg four times daily. Serial plasma cysteamine concentrations were measured for 24 h after the first dose. Participants were reviewed every week for 6 weeks, except at 4 weeks. Plasma cysteamine concentrations were measured 8 h after dosing when reviewed at 1, 2 and 3 weeks and 6 h after dosing when reviewed at 5 weeks. Sputum cysteamine concentration was also quantified at the 5-week assessment. Routine monitoring of clinical microbiology and reporting of speciation and resistance profile of major colonising microorganisms from patients continued as normal, prior to, during where necessary and after the trial by the hospital microbiology laboratory.

$$Formula for calculating FICI \\ FICI = [ \frac{{MIC}_{100} drug A in combination}{{MIC}_{100} drug A alone} ] + [ \frac{{MIC}_{100} drug B in combination}{{MIC}_{100} drug B alone} ] \qquad$$

All media and chemical reagents were purchased from Sigma. Bcc isolates used in this study were either type strains purchased from the American Type Culture Collection (ATCC), the National Collection of Type Cultures (NCTC), of Public Health England, or Deutsche Sammlung von Mikroorganismen and Zellkulturen (DSMZ). Clinical strains were gifted from either Aberdeen Royal Infirmary, or the University of Glasgow Dental School.

### REFERENCES

CLSI. 2012a. Methods for dilution antimicrobial susceptibility tests for bacteria that grow aerobically; Approved Standard - Ninth Edition; M07-A9. Wayne, Pa: Clinical and Laboratory Standards Institute.

Burkhart CG, Burkhart CN, Isham N. 2006. Synergistic antimicrobial activity by combining an allylamine with benzoyl peroxide with expanded coverage against yeast and bacterial species. Br J Dermatol 154:341-344.

CLSI. 2012b. Performance Standards for Antimicrobial Susceptibility Testing; Twenty second Informational Supplement. Clinical and Laboratory Standards Institute, Wayne, PA.

Results

TABLE 19
The predominant pathogens reported in the sputum from three different
patients before and after the Aberdeen clinical trial show a change in
resistance profile

| Patient with | | |
|---|---|---|
| altered clinical | Pre trial | Post trial |
| microbiology | microbiology | microbiology |
| Patient 1 | MRSA | MSSA |
| Patient 2 | PR P. aeruginosa | S P. aeruginosa |
| Patient 3 | Cip^{R} P. aeruginosa | Cip^{S} P. aeruginosa |

(MRSA = Methicillin-resistant Staphylococcus aureus;
MSSA = Methicillin-sensitive Staphylococcus aureus;
PR = pan-resistant;
S = sensitive,
Cip^{R} = ciprofloxacin-resistant;
Cip^{S} = ciprofloxacin sensitive.

TABLE 20
Accompanies FIG. 1 below - The reduction of microbial burden in the
neutropoenic mouse thigh model of infection (illustrated graphically
in FIG. 1) shown as a log10 reduction of cfu/g compared with vehicle
control. Colistin (at 5 mg/kg) was used as a positive control.

| | Log₁₀ reduction |
|---|---|
| Treatment | in cfu/g |
| Vehicle only | 0 |
| Colistin (positive control) | 5.02 |
| [5 mg/kg] | |
| Cysteamine [1.25 mg/kg] | 0.74 |
| Ciprofloxacin [15 mg/kg] | 2.02 |
| Ciprofloxacin + cysteamine | 4.6 |

1. A sulphur-containing compound for use in the prophylaxis or treatment of an infection associated with at least one microbe or microbial strain displaying at least some degree of drug resistance.

2. The sulphur-containing compound for use as claimed in claim 1 wherein the infection is a drug-resistant microbial infection.

3. The sulphur-containing compound for use as claimed in claim 2 wherein the infection is a drug-resistant bacterial infection.

4. The sulphur-containing compound for use as claimed in claim 3 wherein the infection is an antibiotic resistance bacterial infection.

5. The sulphur-containing compound for use as claimed in claim 1 wherein the compound is an aminothiol.

6. The sulphur-containing compound for use as claimed in claim 5 wherein the aminothiol is cysteamine or a derivative thereof

7. The sulphur-containing compound for use as claimed in claim 1 wherein the compound is an organic disulphide.

8. The sulphur-containing compound for use as claimed in claim 7 wherein the organic disulphide is cystamine.

9. A prophylactic or curative treatment of an infection associated with at least one microbe or microbial strain displaying at least some degree of antimicrobial resistance comprising administering to a subject a sulphur-containing compound. Preliminary Amendment

10. A product comprising a first agent being a sulphur-containing compound as claimed in claim 1 and a second agent being an antimicrobial agent.

11. The product as claimed in claim 10 wherein the sulphur-containing agent and the antimicrobial agent are not the same.

12. The product as claimed in claim 10 which does not comprise a peptide.

13. The product as claimed in claim 10 wherein the antimicrobial agent is an antibiotic agent.

14. The product as claimed in claim 10 wherein the antibiotic agent is of the group consisting of aminoglycosides, ansamycins, carbacephem, β-lactams carbapenems, cephalosporins, (including first, second, third, fourth and fifth generation cephalosporins), penicillin, monobactams), glycylcyclines, lincosamides, lipopeptides, macrolides, nitrofurans, oxazolidinones, quinolones, sulfonamides, polypeptides and tetracyclins.

15. The product as claimed in claim 10 wherein the antibiotic agent is a macrolide and/or an aminoglycoside and/or sulphonamide.

16. The product as claimed in claim 10 for use in the prophylaxis or treatment of an infection associated with at least one microbe or microbial strain displaying at least some degree of drug resistance.

17. A prophylactic or curative treatment of an infection associated with at least one microbe or microbial strain displaying at least some degree of antimicrobial resistance comprising administering to a subject a product as claimed in claim 10.

18. A kit comprising a first dosage unit comprising a sulphur-containing compound as claimed in claim 1 and a further dosage unit comprising an antibacterial agent.
